(19) United States
(12) Patent Application Publication
(10) Pub. No.: US 2025/0071492 A1
(43) Pub. Date: Feb. 27, 2025

(54) TINNITUS REMEDIATION WITH SPEECH PERCEPTION AWARENESS

(71) Applicant: Cochlear Limited, Macquarie University, NSW (AU)

(72) Inventors: Remo Albert Gerardus Joseph ARTS, Valkenswaard (NL); Mattheus Johannes Petrus KILLIAN, Mechelen (BE); Bastiaan VAN DIJK, Rosmalen (NL)

(21) Appl. No.: 18/720,814

(22) PCT Filed: Dec. 14, 2022
(86) PCT No.: PCT/IB2022/062248

§ 371 Date: Jun. 17, 2024

Related U.S. Application Data
(60) Provisional application No. 63/292,616, filed on Dec. 22, 2021.

(51) Int. Cl. H04R 25/00; A61B 5/12

(52) U.S. Cl. CPC H04R 25/75; A61B 5/128; H04R 2225/67

(57) ABSTRACT

Presented herein are techniques that set attributes of tinnitus remediation signals delivered to a user based on a predetermined ability of the user to understand target sounds in the presence of background signals. In certain examples, the techniques presented herein provide varying levels of tinnitus relief and target sound perception based on an individual's needs.

### BACKGROUND

### Field of the Invention

The present invention relates generally to hearing devices.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: administering one or more speech perception tests to a user of a hearing device; determining, based on results of the one or more speech perception tests, one or more effects of at least one tinnitus remediation signal on a target sound perception of the user; and configuring the hearing device based on the one or more effects of the at least one tinnitus remediation signal on a target sound perception of the user.

In another aspect, a method is provided. The method comprises: delivering sound signals to a user of a hearing device; and delivering a tinnitus remediation signal to the user of a hearing device simultaneously with the processed sound signal picked up by the microphone of the hearing device, wherein a maximum level of the tinnitus remediation signal is based on a predetermined effect of one or more tinnitus remediation signals on the user's target sound perception.

In another aspect, a method is provided. The method comprises: delivering stimulation signals to a user of a hearing device; delivering a tinnitus remediation signal to the user of the hearing device simultaneously with the stimulation signals; and determining an effect of tinnitus remediation signal on a target sound perception of the user of the hearing device.

In another aspect, one or more non-transitory computer readable storage media are provided. The one or more non-transitory computer readable storage media comprise instructions that, when executed by a processor, cause the processor to: perform the one or more sound perception tests during which stimulation signals are delivered to a user of a hearing device; deliver at least one tinnitus remediation signal to the user of the hearing device simultaneously with the stimulation signals; determine one or more effects of at least one tinnitus remediation signal on a target sound perception of the user; and set one or more operational parameters of the hearing device based on the one or more effects of the at least one tinnitus remediation signal on a target sound perception of the user.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a user wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is an exemplary psychometric curve associated with a Digit Triplet Test administered to a user, in accordance with certain embodiments presented herein;

FIG. 3 is a schematic diagram illustrating a hearing device with which aspects of the techniques presented herein can be implemented;

FIG. 4 is a graph illustrating one implementation of the techniques presented herein;

FIG. 5 is a flowchart of an exemplary method, in accordance with certain embodiments presented herein;

FIG. 6 is a flowchart of another exemplary method, in accordance with certain embodiments presented herein;

FIG. 7 is a flowchart of yet another exemplary method, in accordance with certain embodiments presented herein; and

FIG. 8 is block diagram illustrating a computing device configured to implement aspects of the techniques presented herein.

### DETAILED DESCRIPTION

Tinnitus is the perception of noise or “ringing” in the ears which currently affects an estimated 10-15% of the general population, increasing with age. Tinnitus is a common artefact of hearing loss, but can also be a symptom of other underlying conditions, such as ear injuries, circulatory system disorders, etc. Although tinnitus effects can range from mild to severe, almost one-quarter of those with tinnitus describe their tinnitus as disabling or nearly disabling/incapacitating. Further, tinnitus can deteriorate the quality of a person's life such as, e.g., by negatively impacting the person's sleep quality.

Tinnitus has a particularly high prevalence in hearing-impaired persons and stimulation of the ear, through for instance cochlear implants or other hearing devices (e.g., auditory prosthesis, hearing aids, tinnitus therapy devices, consumer devices that provide audio streaming, consumer headphones, earphones and other listening devices), has shown promising results on tinnitus relief and can be considered as a tinnitus management solution. For example, certain hearing devices can deliver stimulation signals, sometimes referred to herein as “tinnitus remediation signals,” or “tinnitus relief signals,” to a user in order to treat/relieve/remediate tinnitus symptoms. These tinnitus remediation signals can have a number of different forms and underlying objectives. For example, in certain embodiments, the tinnitus remediation signals can be masking signals that are configured to mask/cover the user's tinnitus symptoms (e.g., expose the user to sounds/noises at a loud enough volume that it partially or completely covers the sound of their tinnitus). In other embodiments, the tinnitus remediation signals can be distraction signals that are configured to divert the user's attention from the sound of tinnitus. In other embodiments, the tinnitus remediation signals can be habituation signals that are configured to assist the user's brain in reclassifying tinnitus as an unimportant sound that then can be unconsciously ignored. In still other embodiments, the tinnitus remediation signals can be neuromodulation signals that are configured to minimize the neural hyper- or hypoactivity or to re-model the central auditory system thought to be the underlying cause of tinnitus. In certain embodiments, the tinnitus therapy signals can be any combination of masking signals, distraction signals, habituation signals, and/or neuromodulation signals.

Although the delivery of tinnitus remediation signals can treat a user's tinnitus symptoms, it has also been realized that these tinnitus remediation signals can, in certain examples, negatively affect the user's ability to hear target sounds, such the user's ability to understand speech signals, music signals, alarm signal (e.g., ringing of an alarm bell; signal of an approaching car, etc.), etc. As such, presented herein are techniques that set attributes of tinnitus remediation signals delivered to a user based on a predetermined ability of the user to understand speech in the presence of background signals. In certain examples, the techniques presented herein provide varying levels of tinnitus relief and speech perception based on an individual's needs.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other hearing devices or auditory prosthesis systems that include, e.g., one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by conventional hearing aids or dedicated tinnitus therapy devices and tinnitus therapy device systems. As used herein, the term “hearing device” is to be broadly construed as any device that delivers sound signals to a user in any form, including in the form of acoustical stimulation, mechanical stimulation, electrical stimulation, etc. As such, a hearing device can be a device for use by a hearing-impaired person (e.g., hearing aid, auditory prosthesis, tinnitus therapy devices, etc.) or a device for use by a person with normal hearing (e.g., consumer devices that provide audio streaming, consumer headphones, earphones and other listening devices).

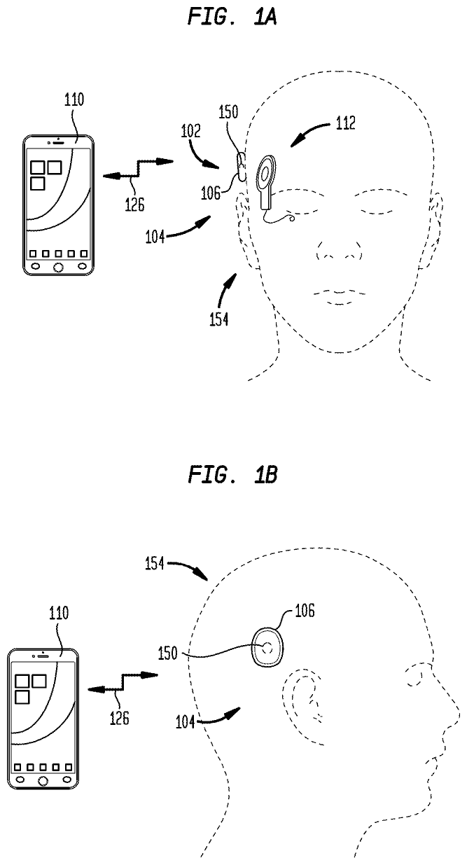

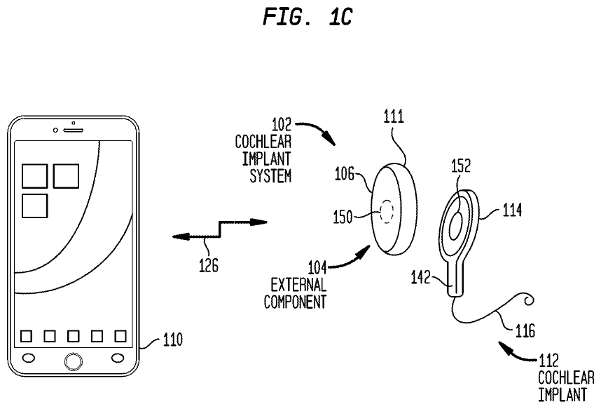

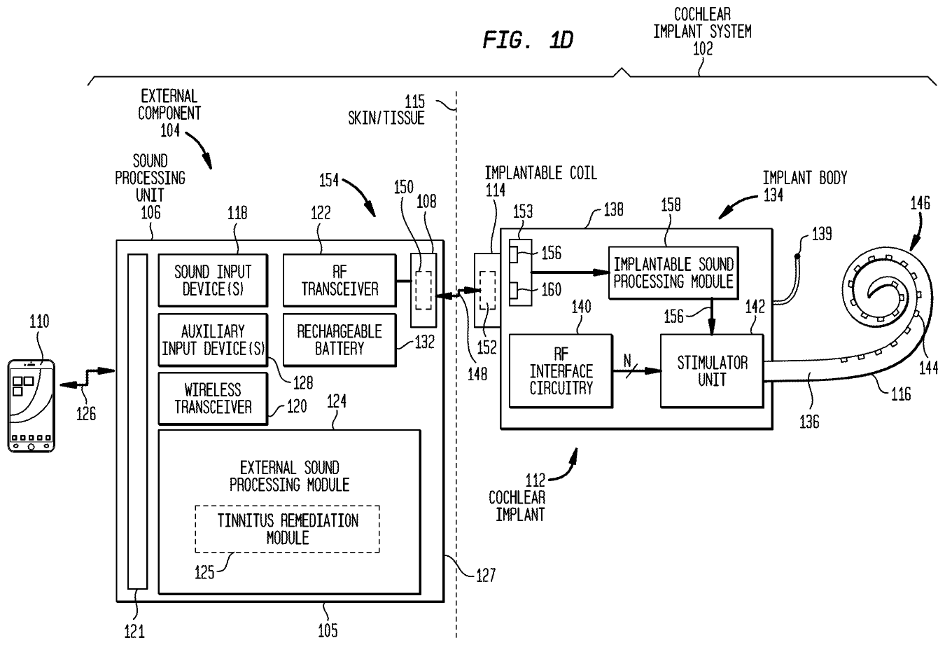

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the user and an implantable component 112 configured to be implanted in the user. In the examples of FIGS. 1A-ID, the external component 104 comprises a sound processing unit 106, while the cochlear implant 112 includes an implantable coil 114, an implant body 134, and an elongate stimulating assembly 116 configured to be implanted in the user's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the user's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 can be used to administer a speech perception or speech perception test, as described further below, and can be configured to implement aspects of the tinnitus remediation techniques presented herein. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 could be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 132, and an external sound processing module 124. The external sound processing module 124 can further include a tinnitus remediation module 125 that, as described further below, can be configured to set attributes of tinnitus remediation signals delivered to a user based on a predetermined ability of the user to understand speech in the presence of background signals. The external sound processing module 124 can comprise, for example, one or more processors and a memory device (memory) that includes sound processing logic and/or tinnitus remediation logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic and/or tinnitus remediation logic stored in memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via, e.g., external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 153, 156, 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 153 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 153, 156, 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals that are provided to the stimulator unit 142. The stimulator unit 142 is configured to utilize the output signals to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 153, 156, 160 in generating stimulation signals for delivery to the user.

As noted above, tinnitus remediation signals can be used to provide tinnitus relief to users of hearing devices, such as hearing aids, cochlear implants, electro-acoustic hearing devices, etc. However, also as noted above, the delivery of tinnitus remediation signals can also negatively affect a user's speech understanding. Presented herein are techniques that effectively achieve a desired level of tinnitus relief while remaining cognizant of the user's perception of target sounds in the presence of tinnitus remediation signals. For example, presented herein are techniques that set one or more attributes of a tinnitus remediation signal delivered to a user so as to limit the impact of the tinnitus remediation signal on the user's perception of target sounds, such as speech perception, music perception, alarm signal detection, etc., collectively and generally referred to herein as the user's “target sound perception.”

More specifically, in accordance with certain embodiments presented herein, a system determines the effect(s) of a tinnitus remediation signal (e.g., a user preferred tinnitus remediation signal or a tinnitus remediation signal measured to be most effective) on the user's target sound perception (e.g., speech perception, alarm signal detection, etc.). In certain embodiments, the effect(s) of the tinnitus remediation signal on the user's target sound perception are used to set a maximum level of the tinnitus remediation signal. The maximum level of the tinnitus remediation signal can be, for example, a maximum level of the tinnitus remediation signal that can be selected by the user, a maximum level of a tinnitus remediation signal automatically selected and/or delivered by a hearing device, etc. In accordance with other embodiments presented herein, the effect(s) of the tinnitus remediation signal on the user's target sound perception are used to set a “warning level” where, if the user selects a level of a tinnitus remediation signal above the warning level, a warning can be generated to the user indicating that the selected level can impact speech perception. In accordance with the techniques presented herein, the maximum level or warning level can be set for, or only be triggered in, specific sound environments (e.g., speech or speech in noise), while allowing higher tinnitus remediation signal levels when there is no speech to be impacted by the tinnitus remediation signal.

In further embodiments, a system determines the effect(s) of two or more tinnitus remediation signals (e.g., user preferred tinnitus remediation signals or tinnitus remediation signals measured to be most effective) on the user's target sound perception. In certain embodiments, the effect(s) of the tinnitus remediation signals on the user's target sound perception are used to select one of the two or more tinnitus remediation signals (or another tinnitus remediation signal) having the least impact on the user's target sound perception.

In accordance with various embodiments, the effect(s) of the tinnitus remediation signal(s) can be determined without considering additional background noise level. However, the above embodiments can each also implemented in a manner that further considers the effects of the tinnitus remediation signal on the user's target sound perception in combination with background noise to determine a combined/total effect on speech perception.

As noted above, aspects of the techniques presented herein include determining the effect(s) of one or more tinnitus remediation signals on the user's target sound perception. In general, the effect(s) of one or more tinnitus remediation signals on the user's target sound perception is determined administering one or more speech perception/intelligibility tests to the user in the presence of the one or more tinnitus remediation signals. That is, in accordance with aspects presented herein, the one or more speech perception tests are performed while delivering a tinnitus remediation signal to the user. The results of the one or more speech perception tests administered in the presence of the one or more tinnitus remediation signals can be used to determine the effects of the one or more tinnitus remediation signals on the user's target sound perception (e.g., speech perception).

The techniques presented herein can make use of a number of different types of speech perception tests and the speech perception tests can be administered in a number of different manners. For example, the techniques presented herein can make use of monaural speech perception tests (e.g., for single sided deaf users) and/or binaural speech perception tests (e.g., for binaural or bilateral hearing device users) that are each administered in the presence of one or more mixed tinnitus remediation signals. In addition, a given speech perception test can be repeated using different tinnitus remediation signals (e.g., sounds or maskers) and the tests can determine which of the different tinnitus remediation signals has the least impact on speech perception, which provides the most satisfactory tinnitus relief for the user (e.g., which is preferred by the user), which tinnitus remediation signal is measured to be the most effective, etc. In one embodiment, the tinnitus remediation signal that least impacts speech perception is chosen for the user, while in other embodiments the user's preferred tinnitus remediation signal is chosen. In a still other embodiment, the tinnitus remediation signal measured to be the most effective is chosen for the user.

In one exemplary embodiment presented herein, the speech perception tests incorporate a Digit Triplet Test (DTT), which is a relatively easy speech perception test that evaluates the user's ability to perceive numbers. In accordance with embodiments presented herein, the DTT speech perception test is conducted while one or more tinnitus remediation signals are also provided to the user. For example, a user is simultaneously provided with a combination of three numbers, i.e., a “digit triplet,” and one or more tinnitus remediation signals. The user then responds by speaking or otherwise indicating the numbers that she perceived. The process of delivering digit triplets to the user and the user responding with the numbers that the user perceived can be repeated. The speech perception percentage can be, e.g., equal the percentage of digits that the user correctly perceived.

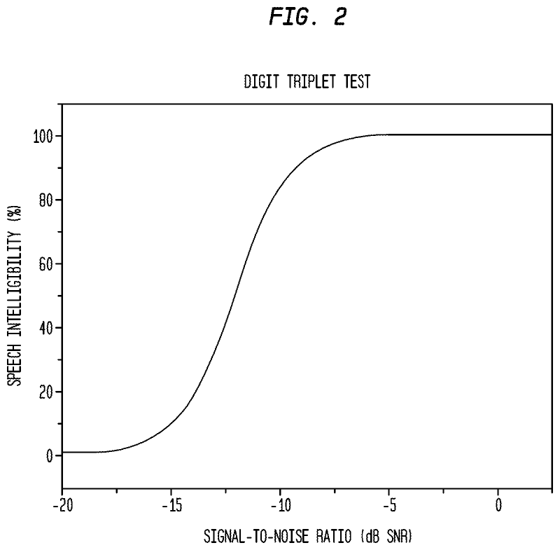

FIG. 2 illustrates a psychometric curve associated with an exemplary speech perception test using a DTT for a particular user. As shown in FIG. 2, the user's speech perception is approximately zero for SNRs between approximately −20 dB and approximately −17.5 dB. From approximately −17.5 dB to approximately −15 dB, the user's speech perception gradually increases from approximately 0% to approximately 10%. From approximately −15 dB to approximately −10 dB, the user's speech perception sharply increases. In particular, at approximately −12.5 dB the user has a speech perception of approximately 50%, and at approximately −10 dB the user has a speech perception of approximately 80%. From approximately −10 dB to approximately −7.5 dB, the user's speech perception gradually increases to approximately 95%, and from approximately −7.5 dB to 0 dB, the user's speech perception even more gradually increases to approximately 100%.

As noted, the DTT is one example of a speech perception test that can be used in accordance with certain embodiments presented herein. It is to be appreciated that any of a number of speech perception tests in any language can also or alternatively be used in embodiments presented herein. For example, the techniques presented herein could use any of the matrix test, sentence test, Hearing in Noise Test (HINT), Speech Recognition in Noise Test (SPRINT), Words in Noise Test (WIN), Diagnostic Rhyme Test (DRT), Diagnostic Medial Consonant Test (DMCT), Diagnostic Alliteration Test (DALT), Modified Rhyme Test (MRT), Phonetically Balanced Word List (PB), Spelling Alphabet Test (SpAT), any speech-in-noise test, any speech discrimination test, or any combination or variation of the foregoing speech recognition/perception where the “noise” is a tinnitus remediation signal.

As noted above, in certain embodiments, the techniques presented can determine a user's “target sound perception,” where the “target sounds” can be speech signals, music signals, alarm signals, etc. As such, it is to be appreciated that the “speech material” used in a speech perception test herein is not limited to numerical digits or even speech. That is, as used herein, the speech material used in the speech perception tests can be any appropriate target sound(s). That is, as used herein, a speech perception test is not limited to delivering only speech in the presence of tinnitus remediation signals, but can also or alternatively delivery other target sounds, such as music signals, alarm signals, etc. Moreover, the speech perception tests could measure temporal/spectral modulation (which correlates highly with speech).

As noted, in accordance with embodiments presented herein, one or more tinnitus remediation signals are delivered to the user during the speech perception test. The one or more tinnitus remediation signals can be selected/determined prior to administering the one or more tinnitus remediation signals to the user during the speech perception test and/or adapted, adjusted, or changed during the speech perception test. a

It is be appreciated that, during a speech perception test, the source of the one or more tinnitus remediation signals is not limited. For example, the tinnitus remediation signal(s) can be provided to a user via her hearing device (e.g., cochlear implant, hearing aid, etc.) or emitted from one or more speakers included in or coupled to, e.g., the hearing device, an external device, etc. Moreover, the one or more tinnitus remediation signals can be presented to the user via electrical stimulation, mechanical stimulation, acoustic stimulation, and/or combinations thereof.

In addition, it is to be appreciated that the compositional characteristics of the tinnitus remediation signal(s) used in a speech perception test are not limited. For example, the tinnitus remediation signal(s) can be sounds occurring in nature (e.g., sounds of a waterfall, ocean waves, or a forest), physiological sounds (e.g., heartbeat), static sounds (e.g., white noise, pink noise, or Brownian noise), etc. The sounds can be reproductions of actual sound recordings from nature or artificial reproductions thereof. Accordingly, the tinnitus remediation signal(s) used can be one or more constant signals, one or more variable signals, or any combination or variation thereof. The user can be able to choose the tinnitus remediation signal(s) or sound(s) that the user prefers. In certain examples, the tinnitus relief signal could be a user selected input sound (e.g. music) which is not predefined by the system (e.g., used during the speech perception alarm signal detection testing).

If a tinnitus remediation signal includes a variable amplitude/level, the speech perception test can be correlated with the tinnitus remediation signal so as to provide the speech material to the user at a time when the level of the tinnitus remediation signal is at or near the maximum level. This correlation ensures that the effect of the tinnitus remediation signal is evaluated at the maximum level (e.g., determine the worst-case or maximum effect of a tinnitus remediation signal on the user's target sound perception). For example, if the tinnitus remediation signal includes crashing ocean waves, the speech material of the speech perception test can be provided to the user at the loudest level, which can be when an ocean wave crashes.

In an alternative embodiment using a tinnitus remediation signal with a variable level, the speech material can be provided to the user at a time when the level of the tinnitus remediation signal is at a predetermined percentage of the maximum level. Further, a determination can be made of the average level (in dB) of the variable tinnitus relief sound, and the speech material can be provided to the user at the time the variable tinnitus relief sound is at the determined average level. Accordingly, each level shown in FIG. 2 can be a maximum, predetermined percentage of a maximum, or average decibel (or SNR) level of a variable tinnitus relief signal.

The curve shown in FIG. 2 is illustrated as a smooth curve. This curve can have been produced by a method of interpolation, extrapolation, curve smoothing, and/or curve fitting that uses a plurality of individual test points as an input. The method of interpolation, extrapolation, curve smoothing, and/or curve fitting is not limited to any particular method and can use, e.g., linear, nonlinear, algebraic, and/or geometric fitting techniques, and can further include a method for identifying and accounting for outliers in the input data.

In accordance with embodiments presented herein, after one or more speech perception tests are complete, the user's hearing device can be configured to operate in one, or various, modes during normal operation. The mode or modes selected for use can take various forms, based on, for example, the results of the speech perception tests (e.g., effect(s) of the tinnitus remediation signal on the user's target sound perception), attributes of the hearing device, user-specific criteria, etc.

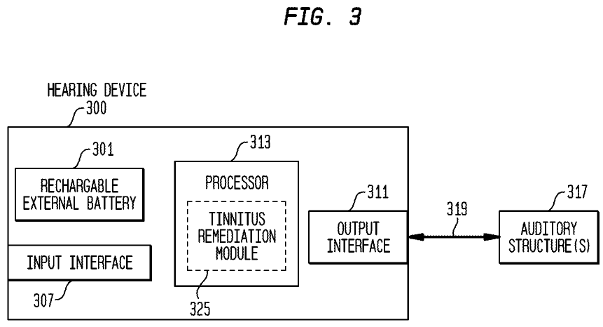

FIG. 3 is a functional block diagram of a hearing device 300 that can be configured to operate in one, or various, modes during normal operation, based on the results of the speech perception tests (e.g., effect(s) of the tinnitus remediation signals). In this example, the hearing device 300 includes a rechargeable battery 301, an input interface 307, an output interface 311, and a processor 313 that includes a tinnitus remediation module 325. As shown, the output interface 311 can be configured to interact with one or more auditory structures 317 of the user of the hearing device 300 via a connection 319. Auditory structure(s) 317 of the user can include, e.g., vibratory structures of the ear such as, e.g., the tympanic membrane, structures of the middle ear (such as the ossicles, malleus, incus, and stapes, the oval window, and the round window), and the cochlea, which includes the auditory nerve endings that can be stimulated electrically with a cochlear implant. These vibratory structures of the ear vibrate to some degree when a person with normal hearing hears sound. But an individual experiencing hearing loss can have reduced or no vibration in one or more of these structures. Specifically, the ossicles in the middle ear can lack the ability to increase the force of sound vibrations to sufficiently stimulate receptor cells in the cochlea. In addition, the ossicles can be broken such that they do not conduct sound vibrations to the oval window and/or the round window of the cochlea. The hearing device 300 can thus serve to partially or fully restore hearing by interacting with one or more auditory structures 317 of the user such as the above-noted vibratory and neuronal structures of the ear. Further, the hearing device 300 can provide tinnitus remediation signals via interaction with the one or more auditory structures 317 of the user.

The connection 319 between the output interface 311 and the hearing device 300 may, e.g., be mechanical, ultrasonic, electrical, acoustic, electro-acoustic, an auditory brain stimulator, or a combination or variation thereof. The hearing device 300 can be, e.g., (1) a cochlear implant having a connection 319 consisting of electrical and/or mechanical stimulators which interact with a user's cochlea; (2) a hearing aid having a connection 319 that amplifies sound; (3) an ultrasonic hearing device having a connection 319 that uses ultrasonic stimulation to create waves/vibrations in brain fluid to vibrate cochlear fluid; (4) a bone conduction hearing device having a connection 319 that transfers sound vibrations to the inner ear through the skull bone; (5) an auditory brainstem implant having a connection 319 that provides sound sensations by stimulating neurons of the cochlear nucleus; or (6) a combination or variation thereof.

The input interface 307 can include components configured to communicate with one or more accessory devices or external devices (not shown in FIG. 3). The input interface 307 can include one or more buttons, touchscreens, or other input devices with which the user can interact. The accessory/external device(s) with which the input interface 307 can communicate can be, e.g., the external device 110 and/or the external component 104 described above with respect to FIG. 1. The accessory/external device(s) can be, e.g., one or more laptop(s), desktop(s), tablet(s), mobile phone(s), wearable device(s), computer(s), or other computing device(s).

As noted, the hearing device 300 can be configured to provide customized tinnitus relief signals for the specific individual using, wearing, or receiving the hearing device 300, using the effect(s) of the tinnitus remediation signal on the user's target sound perception. That is, as noted above, the results of the speech perception tests (e.g., effect(s) of the tinnitus remediation signal on the user's target sound perception) can be used to program the hearing device 300 to operate in one, or various, modes during normal operation. The mode or modes selected for use can take various forms, based on, for example, the results of the speech perception tests (e.g., effect(s) of the tinnitus remediation signal on the user's target sound perception), attributes of the hearing device 300, user-specific criteria, etc.

In certain embodiments, the results of the speech perception tests (e.g., effect(s) of the tinnitus remediation signal on the user's target sound perception) are used to set a maximum level of a tinnitus remediation signal delivered to a user via the hearing device 300. The maximum level of the tinnitus remediation signal can be, for example, a maximum level of the tinnitus remediation signal that can be selected by the user, a maximum level of a tinnitus remediation signal automatically selected and/or delivered by a hearing device 300, etc. The maximum level could be set the same for all use conditions (e.g., all sound environments) experienced by the user, or could vary based on the use conditions experienced by the user.

For example, hearing device 300 can, in certain embodiments, be configured to “classify” a user's sound (ambient) environment into one of a plurality of “sound classes” or “sound environments.” These sound classes/environments can include, for example, “speech,” “speech-in-noise,” “noise,” “quiet,” “wind,” “music,” etc., or subsets thereof. In such embodiments, the techniques presented herein can configure the hearing device 300 to use a different maximum level of the tinnitus remediation signal in different sound environments (different sound classes), use different types of tinnitus remediation signals in different sound environments, slowly switched on/off based on the environment, etc. In other words, in certain embodiments, the maximum level of the tinnitus remediation signal, the type of the tinnitus remediation signal, and/or other attributes of a delivered tinnitus remediation signal can depend on the sound classification determined by the hearing device 300 (e.g., automatically activate delivery of tinnitus remediation signals when the sound class is “quiet”).

The hearing device 300 could be pre-programmed with a specific maximum level, type, or other attributes of the tinnitus remediation signal to be used in sound class and/or the specific maximum level, type, or other attributes of the tinnitus remediation signal could be determined or adjusted dynamically by the hearing device 300 using feedback from the user.

In accordance with other embodiments presented herein, the effect(s) of the tinnitus remediation signal on the user's target sound perception are used to set a “warning level” where, if the user selects a level of a tinnitus remediation signal above the warning level, a warning can be generated to the user indicating that the selected level may impact target sound perception (e.g., speech perception, alarm signal detection, etc.). Similar to the above, accordance with the techniques presented herein, the warning level can be set for, or only be triggered in, specific sound environments/classes (e.g., speech or speech in noise), while allowing higher tinnitus remediation signal levels when there is no speech to be impacted by the tinnitus remediation signal. The warning could be delivered via the hearing device 300 and/or another device operating with the hearing device (e.g., a mobile phone wireless connected to the hearing device).

In accordance with certain embodiments presented herein, the effect(s) of the tinnitus remediation signal on the user's target sound perception are used to program the hearing device 300 to operate in a mode that provides increased tinnitus relief with the tradeoff of decreased speech perception ability. Another mode can provide increased speech perception ability with the tradeoff of decreased tinnitus relief. A third mode can provide moderate tinnitus relief while also providing moderate speech perception ability. In one embodiment, there are three modes that provide high, moderate, and low tinnitus relief, where the high, moderate, and low tinnitus relief modes are respectively associated with a low, moderate, and high speech perception ability. For example, in certain embodiments, the system can use a certain SNR level for the tinnitus remediation and the “high” setting is a setting in which (speech will be affected below 70 dB SPL, the “medium” setting is a setting in which speech will be affected if it's below 60 dB, and the “low” setting is a setting in which speech will only be affected when it's below 50 dB. It is to be appreciated that these values are merely illustrative and that the number of configurable modes is not particularly limited, and any number of modes can be configured that provide varying levels of tinnitus relief and speech perception ability.

In one embodiment, each mode of the plurality of modes are associated with a specific level/percentage of target sound perception, recognition, or intelligibility. For example, a high tinnitus relief mode can be associated with a very low target sound perception percentage, e.g., 10%; a moderate tinnitus relief mode can be associated with a moderate speech perception percentage, e.g., 50%; and a low tinnitus relief mode can be associated with a high speech perception percentage, e.g., a percentage at or near 100%. The specific target sound perception percentage/level for each of the plurality of modes is not particularly limited, and any percentage/level can be set for each of the plurality of modes. In one embodiment, the user can be able to choose any specific percentage of target sound perception ability between zero and one hundred percent, and the hearing device 300 will respond with a corresponding tinnitus relief mode. Instead of a specific percentage, each mode of the plurality of modes can alternatively associated with a plurality of ranges of target sound perception ability. For example, a high, moderate, and low tinnitus relief mode can be respectively associated with target sound perception percentage ranges of, e.g., 0% to 10%; 45% to 55%; 90% to 100%.

The tinnitus remediation signal provided to the user may not be the same for each of the plurality of operational modes. In this regard, there can be sound(s) that provide more tinnitus relief to the user than other sound(s). As such, in addition to specific level(s) of target sound perception, the tinnitus remediation signal can be associated with each of the plurality of operational modes. For example, one tinnitus remediation signal that least impacts target sound perception ability can be used for the low tinnitus relief but high target sound perception ability mode, and another tinnitus remediation signal that provides the most tinnitus relief can be used for the high tinnitus relief but low target sound perception ability mode.

The manner in which the target sound perception ability is configured with respect to each of the plurality of modes is also not limited. The user of the hearing device 300 can interact with an external or accessory device such as a smart phone, tablet, desktop, wearable device, or other computer to configure the tinnitus relief and target sound perception ability for each of the plurality of modes. For example, the speech perception test can be administered via the user's interaction with the external or accessory device. Accordingly, presence of a health care provider is not necessarily required for the user to configure the plurality of modes of the hearing device 300. In the alternative, the configuration of the plurality of modes can be configured by or with the help of a health care provider. For example, a health care provider can oversee or facilitate the speech perception test(s) and the configuration of the level(s) of speech perception for each of the plurality of modes.

As noted, the user can be made aware of each level of speech perception for each of the plurality of modes such that the user can suitably control his or her desired level of tinnitus relief, while remaining cognizant of his or her level of speech perception ability.

After the hearing device 300 has been configured with one or more user-specific levels of target sound perception ability and corresponding levels of tinnitus relief, the hearing device 300 can further be configured to operate at a particular mode. In one embodiment, the operational mode is manually selected via input from a user, and in another embodiment, the operational mode automatically changes.

With manual selection, the hearing device 300 can receive an input via input interface 307 to change the operational mode. As noted, the mechanism by which the input interface 307 of the hearing device 300 receives an input is not particularly limited. For example, the input can be effectuated via interaction with one or more buttons, touchscreens, or other input devices with which the user can interact. The buttons, touchscreens, or other input devices can be located on the hearing device 300 itself or on one or more accessory or external devices. In one embodiment, the user interacts with a smart phone, smart watch, or other wearable device to change the operational mode of the hearing device 300. The user may, e.g., press a screen of buttons, make gestures, or speak commands to change the operational mode of the hearing device 300.

Accordingly, the user can be able to control his or her level of tinnitus relief and target sound perception ability in various circumstances. There can be a circumstance in which tinnitus relief is more important to the user than target sound perception ability, other circumstance in which target sound perception ability is more important to the user than tinnitus relief, and yet another circumstance in which the user can desire both target sound perception ability and tinnitus relief. For example, if the user is in an environment, such as work or school, in which understanding a target sound, such as speech, is more important to the user than tinnitus relief, the user can facilitate changing the operational mode to a mode having low tinnitus relief and high speech perception ability. Alternatively, if the user is in an environment, such as at home or in a quiet environment, in which understanding speech is not as important to the user as tinnitus relief, the user can provide facilitate changing the operational mode to a mode having high tinnitus relief and low speech perception ability. Further, if the user is in an environment, such as a social environment, in which the user desires both tinnitus relief and some level of target sound perception ability, the user facilitate changing the operational mode to a mode having moderate tinnitus relief and moderate target sound perception ability (e.g., the ability to detect alarm signals while sleeping or in quiet environments). As noted, the number of operational modes is not limited, and the user can be able to choose from any number of modes to achieve the desired level of tinnitus relief and/or level of target sound perception ability.

With automatic selection, the hearing device 300 can automatically change the level, type, or other attributes of tinnitus remediation signals provided to the user without user input based on, for example, attributes of the sound signals received by the hearing device. In certain embodiments, the hearing device 300 could be initially configured with the tinnitus remediation signals adjustments (e.g. in terms of level, type, etc.) and the adjustment triggers (e.g., which attributes of the sound signals cause the tinnitus remediation signals adjustments). In further embodiments, the hearing device 300 or a co-operating external device could implement a machine-learning process (e.g., part of the tinnitus remediation module 325) to dynamically adapt the tinnitus remediation signals adjustments (e.g. in terms of level, type, etc.) and/or the adjustment triggers (e.g., which attributes of the sound signals cause the tinnitus remediation signals adjustments). In certain such embodiments, the machine-learning process could adapt the tinnitus remediation signals adjustments and/or the adjustment triggers based on feedback from the user (e.g., track adjustments made manually by the user via input interface 307) in order to optimize the delivered tinnitus remediation signal(s) for the user.

In certain embodiments, the background noise or the sound level of the ambient environment can provide some level of tinnitus relief to the user and/or can affect the level of target sound perception of the user. Therefore the amount of tinnitus relief provided to the user by the hearing device 300 and the tinnitus relief provided by the background noise can collectively impact the level of target sound perception of the user.

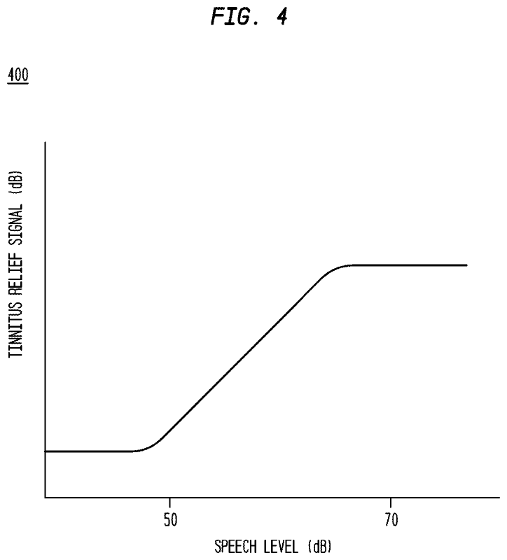

FIG. 4 illustrates one example implementation in accordance with certain embodiments presented herein. In this example, the relationship between the level of speech and the level of a tinnitus remediation signal received by the user is adaptive. The horizontal axis indicates a decibel level of speech provided to a user, and the vertical axis indicates a decibel level of tinnitus remediation signal provided to the user. In other embodiments, the system can set the tinnitus remediation signal to a level while being agnostic to the speech level (e.g., multiple curves for different speech levels and the higher the speech level the less the speech perception is affected by the tinnitus remediation signal).

In one embodiment, a specific speech perception ability level is maintained notwithstanding the noise level of the ambient environments in which the user is located. The sound level of the ambient environment in which the user is located can be measured, e.g., by a microphone within or coupled to the hearing device 300. In one embodiment, when the sound level of the ambient environment increases, the amount of tinnitus relief supplied by the hearing device 300 decreases; and when the sound level of the ambient environment decreases, the amount of tinnitus relief supplied by the hearing device 300 increases. As such, automatic changes to the amount of tinnitus relief supplied by the hearing device 300 can maintain a specific level of speech perception ability even if the background noise or sound level of the ambient environment changes.

As noted, in certain embodiments, the extent to which the amount of tinnitus relief supplied by the hearing device 300 changes can be determined via speech perception test(s). For example, the speech perception test(s) can be administered in a variety of tests that simultaneously provide speech material, tinnitus relief signals (provided by the hearing device 300), and predetermined levels of background noise. If the speech perception test(s) are administered in a controlled environment such as the user's home or an office of a health care provider, the specific levels of background noise provided during the speech perception test(s) can be emulations of predetermined levels of noise similar to those that can occur while the user/patient is outside of the controlled environment. Additionally, since the user can quickly and easily perform speech perception test(s), e.g., by using his or her smart phone or wearable device to administer the speech perception test(s), the user can administer the speech perception test(s) in actual ambient environments in which the user is located. As such, the system can empirically create and use a data repository of speech perception abilities based on the varying levels of background noise and levels of tinnitus relief signals provided by the hearing device 300 that are specific to a particular user.

Further, the system can use one or more software models that predict the extent to which speech perception ability can be impacted by background noise(s). The model(s) can use the background noise as input and output the level of tinnitus relief that should be supplied by the hearing device 300 to maintain a specific level of speech perception. In any case, the system can be configured such that predetermined levels of speech perception are maintained regardless of the level of loudness of the user's ambient environment.

In another embodiment, the speech perception ability can change when the user changes between environments having different levels of background noise. In one embodiment, the system is configured to determine the level of background noise in the environment in which the user is located, and provide a level of tinnitus relief suitable for the determined level of background noise. A determination can be made that the user is in a quiet environment, in a noisy environment, or in an environment of moderate noise level. In one embodiment, if the background noise (i.e., the sound level of the ambient environment) is determined to be below a low threshold level, it is determined the user is in a quiet environment; if the background noise is determined to be above a high threshold level, it is determined the user is in a noisy environment; and if the background noise is determined to be between the low and high threshold levels, it is determined the user is in an environment of moderate noise level.

The method by which the level of speech perception changes based on the background noise level of the user's environment, is not particularly limited. In one embodiment, if the user is determined to be in a quiet environment, the hearing device 300 can automatically change the operational mode to provide a high level of tinnitus relief, which consequently has a low level of speech perception ability. If the user is determined to be in a noisy environment, the hearing device 300 can automatically change the operational mode to provide a low level of tinnitus relief in order to provide the user with a high level of speech perception ability. If the user is determined to be in an environment of moderate noise level, the hearing device 300 can automatically change the operational mode to provide a moderate level of tinnitus relief and a moderate speech perception ability level. The number of speech perception ability/tinnitus relief levels is not limited to three and can be any number or a %. Further, the system can provide a smooth and continuous tinnitus remediation signal that can slowly change from one level to the next so as to avoid abruptly changing between discrete levels (e.g., ensure slow fluctuations of the tinnitus remediation signals to ensure the tinnitus treatment remains acceptable with only slowly adapting signals). Accordingly, the system conveniently change the tinnitus relief level and speech perception level without the user having to provide input to the system. The tinnitus relief/speech perception levels provided, which change as a function of background noise level, can be customized by the user or a health care provider. As such, the user can automatically achieve the desired optimal tinnitus relief and speech perception ability.

The user can be made aware of the level of speech perception ability that is associated with the current operational mode. In one embodiment, when the level of tinnitus relief is changed, the user can receive an indication that communicates the new level of speech perception ability to the user. For example, the user can receive a warning if their speech perception ability has been lowered, especially if the speech perception ability has been lowered below a predetermined speech perception ability threshold.

The method by which the indication or warning of the level of speech perception ability is provided to the user is not limited. For example, the hearing device 300 can provide the user a spoken message, e.g., that tells the user their speech perception ability has changed. The spoken message can further indicate the specific level or percentage of speech perception of the new operational mode. In addition to or instead of being a spoken message, the indication can be one or more recognizable sounds and/or tactical feedback. In addition to or instead of providing the indication via the hearing device 300, the indication can be provided to an external or accessory device, such as the user's smart phone, smart watch, or other wearable device. Further, the user can be able to interact with the hearing device, external device, or accessory device to obtain information regarding the speech perception ability associated with the current operational mode.

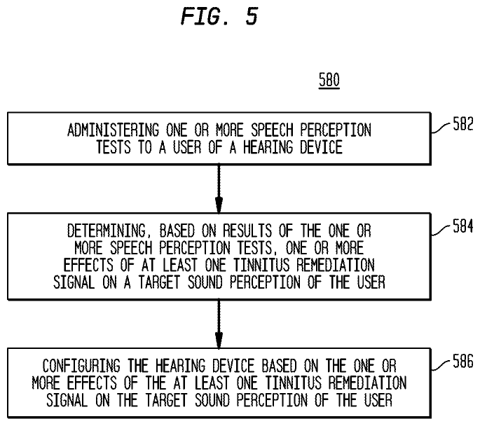

FIG. 5 is a flowchart of an example method 580, in accordance with certain embodiments presented herein. Method 580 begins at 582 where one or more speech perception tests are administered to a user of a hearing device. At 584, the results of the one or more speech perception tests are used to determine one or more effects of at least one tinnitus remediation signal on a speech perception of the user. At 586, the hearing device is configured (e.g., one or more operational parameters or settings of the hearing device are set, selected, adjusted, or otherwise determined) based on the one or more effects of the at least one tinnitus remediation signal on a speech perception of the user.

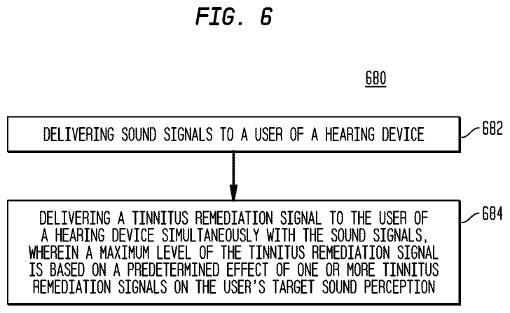

FIG. 6 is a flowchart of an example method 680, in accordance with certain embodiments presented herein. Method 680 begins at 682 where sound signals are delivered to a user of a hearing device. At 684, a tinnitus remediation signal is delivered to the user of a hearing device simultaneously with the sound signal, wherein a maximum level of the tinnitus remediation signal is based on a predetermined effect of one or more tinnitus remediation signals on the user's target sound perception.

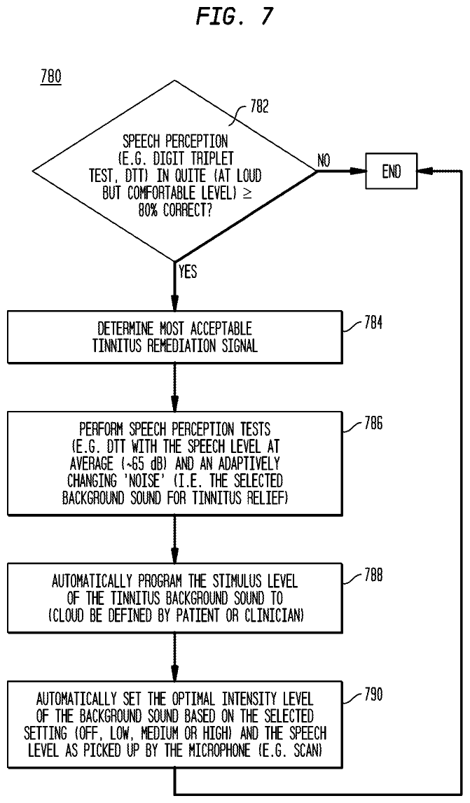

FIG. 7 is a more detailed flowchart of one specific method 780, in accordance with certain embodiments presented herein. Method 780 begins at 782 where the speech perception of a hearing device user suffering from tinnitus is evaluated. In one specific arrangement, a DTT is performed in quiet to determine whether the user's target sound perception is greater than a threshold level (e.g., at least 80% correct). If the user's target sound perception is not greater than the threshold, then the method 780 ends. However, if the user's target sound perception is not greater than the threshold, the method proceeds to 784 where the most acceptable tinnitus remediation signal for the user is determined.

At 786, one or more speech perception tests (adaptive speech in noise tests), such as a DTT) with the speech level at average (˜65 dB) and an adaptively changing ‘noise’ (i.e. the selected background sound for tinnitus relief). These one or more speech perception tests can be used to determine, for example, worst SNR with Speech Reception Threshold (SRT) of 95% of speech perception score from above screening test correct, Worst SNR with SRT of 90% of speech perception score from above screening test correct, Worst SNR with SRT of 85% of speech perception score from above screening test correct.

At 788, the hearing device is configured/programmed automatically program the stimulus level of the tinnitus background sound to (could be defined by patient or clinician) be, for example, OFF (no tinnitus background sound), LOW (background stimulus level automatically adjusted with SCAN to obtain the SNR with SRT of 95% correct from above test), MEDIUM (background stimulus level automatically adjusted with SCAN to obtain the SNR with SRT of 90% correct from above test), HIGH (background stimulus level automatically adjusted with SCAN to obtain the SNR with SRT of 85% correct from above test). At 790, the solution automatically sets the optimal intensity level of the background sound based on the selected setting (OFF, LOW, MEDIUM or HIGH) and the speech level as picked up by the microphone (SCAN).

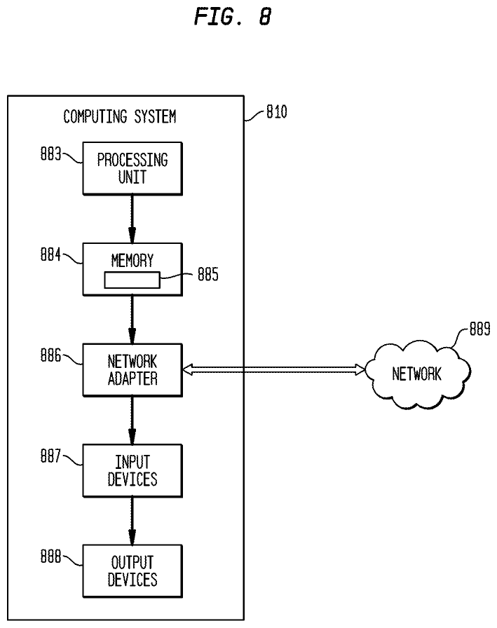

As noted above, aspects of the techniques presented make use of a computing device, such as a fitting system, mobile phone, tablet computer, etc. FIG. 8 illustrates an example of a suitable computing device 810 with which one or more of the disclosed examples can be implemented. Computing devices, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like. The computing system 810 can be a single virtual or physical device operating in a networked environment over communication links to one or more remote devices, such as an implantable medical device or implantable medical device system.

In its most basic configuration, computing system 810 includes at least one processing unit 883 and memory 884. The processing unit 883 includes one or more hardware or software processors (e.g., Central Processing Units) that can obtain and execute instructions. The processing unit 883 can communicate with and control the performance of other components of the computing system 810.

The memory 884 is one or more software or hardware-based computer-readable storage media operable to store information accessible by the processing unit 883. The memory 884 can store, among other things, instructions executable by the processing unit 883 to implement applications or cause performance of operations described herein, as well as other data. The memory 884 can be volatile memory (e.g., RAM), non-volatile memory (e.g., ROM), or combinations thereof. The memory 884 can include transitory memory or non-transitory memory. The memory 884 can also include one or more removable or non-removable storage devices. In examples, the memory 884 can include RAM, ROM, EEPROM (Electronically-Erasable Programmable Read-Only Memory), flash memory, optical disc storage, magnetic storage, solid state storage, or any other memory media usable to store information for later access. In examples, the memory 884 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 884 can include wired media such as a wired network or direct-wired connection, and wireless media such as acoustic, RF, infrared and other wireless media or combinations thereof. In certain embodiments, the memory 884 comprises speech perception testing logic 885 that, when executed, enables the processing unit 883 to perform aspects of the techniques presented (e.g., administer speech perception tests, determine effects of tinnitus remediation signals on a user's target sound perception, etc.).

In the illustrated example, the system 810 further includes a network adapter 886, one or more input devices 887, and one or more output devices 888. The system 810 can include other components, such as a system bus, component interfaces, a graphics system, a power source (e.g., a battery), among other components.

The network adapter 886 is a component of the computing system 810 that provides network access (e.g., access to at least one network 889). The network adapter 886 can provide wired or wireless network access and can support one or more of a variety of communication technologies and protocols, such as ETHERNET, cellular, BLUETOOTH, near-field communication, and RF (Radiofrequency), among others. The network adapter 886 can include one or more antennas and associated components configured for wireless communication according to one or more wireless communication technologies and protocols.

The one or more input devices 887 are devices over which the computing system 810 receives input from a user. The one or more input devices 887 can include physically-actuatable user-interface elements (e.g., buttons, switches, or dials), touch screens, keyboards, mice, pens, and voice input devices, among others input devices.

The one or more output devices 888 are devices by which the computing system 810 is able to provide output to a user. The output devices 888 can include, displays, speakers, and printers, among other output devices.

It is to be appreciated that the arrangement for computing system 810 shown in FIG. 8 is merely illustrative and that aspects of the techniques presented herein may be implemented at a number of different types of systems/devices. For example, the computing system 810 could be a laptop computer, tablet computer, mobile phone, surgical system, etc.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method comprising:
administering one or more speech perception tests to a user of a hearing device;
determining, based on results of the one or more speech perception tests, one or more effects of at least one tinnitus remediation signal on a target sound perception of the user; and
configuring the hearing device based on the one or more effects of the at least one tinnitus remediation signal on the target sound perception of the user.

2. The method of claim 1, wherein administering the one or more speech perception tests to the user comprises:
administering one or more Digit Triplet Tests (DTTs) to the user.

3. The method of claim 1, wherein administering the one or more speech perception tests to the user comprises:
administering one or more monaural speech perception tests.

4. The method of claim 1, wherein administering the one or more speech perception tests to the user comprises:
administering one or more binaural speech perception tests.

5. The method of claim 1, wherein administering the one or more speech perception tests to the user comprises:
administering two or more different speech perception tests to the user in a presence of one or more tinnitus remediation signals.

6. The method of claim 1, wherein administering the one or more speech perception tests to the user of the hearing device comprises:
administering at least a first speech perception test to the user;
while administering the first speech perception test, delivering a first tinnitus remediation signal to the user;
administering at least a second speech perception test to the user; and
while administering the second speech perception test, delivering a second tinnitus remediation signal to the user.

7. The method of claim 6, wherein configuring the hearing device based on the one or more effects of the at least one tinnitus remediation signal on the target sound perception of the user comprises:
determining which of the first tinnitus remediation signal or the second tinnitus remediation signal has the least impact on the user's target sound perception; and
configuring the hearing device to deliver only first tinnitus remediation signal or the second tinnitus remediation signal to having the least impact on the user's target sound perception.

8. The method of claim 1, wherein configuring the hearing device based on the one or more effects of the at least one tinnitus remediation signal on the target sound perception of the user comprises:
setting a maximum level of a tinnitus remediation signal delivered to the user.

9. The method of claim 8, wherein setting the maximum level of the tinnitus remediation signal delivered to the user comprises:
setting the maximum level configured to be activated only in one or more specific sound environments.

10. The method of claim 1, wherein configuring the hearing device based on the one or more effects of the at least one tinnitus remediation signal on the target sound perception of the user comprises:
setting a type of tinnitus remediation signal delivered to the user.

11. The method of claim 10, wherein setting the type of the tinnitus remediation signal delivered to the user comprises:
configuring the hearing device to use a selected type of tinnitus remediation signal in one or more specific sound environments.

12. The method of claim 10, wherein setting the type of the tinnitus remediation signal delivered to the user comprises:
configuring the hearing device to use different types of tinnitus remediation signals in different sound environments.

13. The method of claim 1, wherein configuring the hearing device based on the one or more effects of the at least one tinnitus remediation signal on the target sound perception of the user comprises:
setting a warning level for a first tinnitus remediation signal where, if the user selects a level of the first tinnitus remediation signal above the warning level, a warning can be generated to the user indicating that the selected level of the selected tinnitus remediation signal is likely to impact target sound perception.

14. The method of claim 1, wherein determining the one or more effects of at least one tinnitus remediation signal on the target sound perception of the user comprises:
determining the one or more effects of at least one tinnitus remediation signal on the target sound perception of the user with respect to additional background noise levels.

15. The method of claim 1, wherein determining, based on results of the one or more speech perception tests, one or more effects of at least one tinnitus remediation signal on a target sound perception of the user comprises:
determining, based on results of the one or more speech perception tests, one or more effects of at least one tinnitus remediation signal on a speech perception of the user.

16. The method of claim 1, wherein determining, based on results of the one or more speech perception tests, one or more effects of at least one tinnitus remediation signal on a target sound perception of the user comprises:
determining, based on results of the one or more speech perception tests, one or more effects of at least one tinnitus remediation signal on an alarm signal detection of the user.

17. The method of claim 1, wherein administering one or more speech perception tests to a user of a hearing device comprises:
delivering sound signals to a user of a hearing device; and
delivering a tinnitus remediation signal to the user of a hearing device simultaneously with processed sound signal picked up by a microphone of the hearing device,
wherein a maximum level of the tinnitus remediation signal is based on a predetermined effect of one or more tinnitus remediation signals on the target sound perception of the user.

18. The method of claim 17, further comprising:
receiving an input from at least one of the user or a health care professional; and
changing the maximum level of the tinnitus remediation signal delivered to the user of the hearing device based on the input.

19. (canceled)

20. The method of claim 17, further comprising:
receiving sounds from an ambient sound environment of the user;
determining a sound classification for the ambient sound environment; and
setting the maximum level of the tinnitus remediation signal further based on the sound classification of the ambient sound environment.

21. The method of claim 1, wherein the tinnitus remediation signal is an electrical stimulation signal.

22. The method of claim 1, wherein the at least one tinnitus remediation signal is an acoustic stimulation signal.

23. The method of claim 1, wherein the at least one tinnitus remediation signal is a combination of an acoustic stimulation signal and an electrical stimulation signal.

24. The method of claim 1, wherein the at least one tinnitus remediation signal is a mechanical stimulation signal.

25. (canceled)

26. (canceled)

27. One or more non-transitory computer readable storage media comprising instructions that, when executed by a processor, cause the processor to:
perform one or more sound perception tests during which stimulation signals are delivered to a user of a hearing device;
deliver at least one tinnitus remediation signal to the user of the hearing device simultaneously with the stimulation signals;
determine one or more effects of at least one tinnitus remediation signal on a target sound perception of the user; and
set one or more operational parameters of the hearing device based on the one or more effects of the at least one tinnitus remediation signal on a target sound perception of the user.

28. The one or more non-transitory computer readable storage media of claim 27, wherein the instructions operable to perform the one or more sound perception tests comprise instructions that, when executed, cause the processor to:
perform one or more Digit Triplet Tests (DTTs).

29. The one or more non-transitory computer readable storage media of claim 27, wherein the instructions operable to perform the one or more sound perception tests comprise instructions that, when executed, cause the processor to:
perform one or more monaural speech perception tests.

30. The one or more non-transitory computer readable storage media of claim 27, wherein the instructions operable to perform the one or more sound perception tests comprise instructions that, when executed, cause the processor to:
perform one or more binaural speech perception tests.

31. The one or more non-transitory computer readable storage media of claim 27, wherein the instructions operable to perform the one or more sound perception tests comprise instructions that, when executed, cause the processor to:
perform two or more different speech perception tests in a presence of the at least one tinnitus remediation signal.

32. The one or more non-transitory computer readable storage media of claim 27, wherein the instructions operable to set one or more operational parameters of the hearing device comprise instructions that, when executed, cause the processor to:
set a maximum level of a tinnitus remediation signal delivered to the user.

33. The one or more non-transitory computer readable storage media of claim 32, wherein the instructions operable to set the maximum level of the tinnitus remediation signal delivered to the user comprise instructions that, when executed, cause the processor to:
set the maximum level configured to be activated only in one or more specific sound environments.

34. The one or more non-transitory computer readable storage media of claim 27, wherein the instructions operable to set one or more operational parameters of the hearing device comprise instructions that, when executed, cause the processor to:
set a type of tinnitus remediation signal delivered to the user.

35. The one or more non-transitory computer readable storage media of claim 34, wherein the instructions operable to set the type of the tinnitus remediation signal delivered to the user comprise instructions that, when executed, cause the processor to:
configure the hearing device to use a selected type of tinnitus remediation signal in one or more specific sound environments.

36. The one or more non-transitory computer readable storage media of claim 34, wherein the instructions operable to set the type of the tinnitus remediation signal delivered to the user comprise instructions that, when executed, cause the processor to:
configure the hearing device to use different types of tinnitus remediation signals in different sound environments.

37. The one or more non-transitory computer readable storage media of claim 27, wherein the instructions operable to set one or more operational parameters of the hearing device comprise instructions that, when executed, cause the processor to:
set a warning level for a first tinnitus remediation signal where, if the user selects a level of the first tinnitus remediation signal above the warning level, a warning can be generated to the user indicating that the selected level of the selected tinnitus remediation signal is likely to impact target sound perception.
